(19) United States
(12) Patent Application Publication
(10) Pub. No.: US 2014/0034067 A1
(43) Pub. Date: Feb. 6, 2014

(54) Tube To Prevent Thumb Sucking

(71) Applicant: Margo K. Brilliant, North Miami Beach, FL (US)

(72) Inventor: Margo K. Brilliant, North Miami Beach, FL (US)

(21) Appl. No.: 13/959,988

(22) Filed: Aug. 6, 2013

Related U.S. Application Data
(60) Provisional application No. 61/679,869, filed on Aug. 6, 2012.

(51) Int. Cl. A61F 5/50

(52) U.S. Cl. CPC A61F 5/50
USPC 128/880

(57) ABSTRACT

The present invention is an apparatus to prevent thumb sucking for attachment to a user's arm having an elongated tubular sleeve of a soft pliable material with a closed end and an open end, and a soft pliable material liner within the outer elongated tubular sleeve. The user will have the tubular sleeve placed over the hand and forearm of the user, so that the user will not be able to thumb suck since the tubular sleeve prevents the user from having access to the user's thumb. The tublular sleeve having one or more icons thereon to encourage the user to be receptive to the placement of the tubular sleeve on the user.

### CROSS-REFERENCE TO RELATED APPLICATIONS

This application claims priority to U.S. Provisional Application No. 61/679,869 filed on Aug. 6, 2012 entitled Tube To Prevent Thumb Sucking, the disclosures of which is incorporated herein, by reference, in its entirety.

### BACKGROUND OF THE INVENTION

The present invention relates to an apparatus used to prevent sucking of the thumb. According to Wikipedia®, the free on-line encyclopedia, thumb sucking is a behavior found in humans, and other primates. It usually involves placing the thumb of a hand into the mouth and rhythmically repeating sucking contact for a prolonged duration. It is considered to be soothing and therapeutic for the person. Some children and adults will retain the habit, which amongst other issues, can cause severe dental problems.

### BRIEF SUMMARY OF THE INVENTION

The present invention is an apparatus that prevents a thumb sucker from sucking the thumb or other piece of the skin. The apparatus is an elongated tube or tubular sleeve having a first closed end and a second open end. An interior liner is preferably used within the elongated tube. The tube is designed primarily for children and therefore is intended to be friendly to the user. The first end will therefore have associated with it an icon such as an animal. The second end of the elongated tube is closable with a string, elastic or other closure device. In use a child will insert their hand, the thumb sucking hand, and at least their forearm, into the tube such that the fingers and thumb will be at the first end of the tube. The second end of the tube will be approximately on the forearm of the user's arm, preferably medial to the elbow, and a parent or other person will secure the tube in place by tying the second end securely in position on the forearm so that the child or user cannot remove the tube to place the thumb in the user's mouth. In summary, the child's hand is within the tube so that the child will be denied access to the child's thumb and prevented from sucking the thumb. The tube is a barrier to prevent thumb sucking. The present invention, the tube is designed to look like a friendly animal or other icon that the child will be comfortable with and not protest when the tube is placed on the child's hand and arm, and while the child is wearing the tube. When the child's hand is within the tube at the first end, the thumb cannot be isolated to be sucked since it is within the tube.

### BRIEF DESCRIPTION OF THE DRAWINGS

FIG. 1 is a top plan view of the apparatus of the present invention.

FIG. 2 is sectional view of the tube of the present invention shown with the hand and forearm of the user in place. The tube is shown in sectional view with cross hatching to show the relationship of the hand within the tube.

FIG. 3 is a partial view of the tube around a user's arm with the string tied in a bow.

FIG. 4 is a side view of the tube with an animal icon attached to the tube with a portion in section to show an alternate attachment using an elastic string.

### DETAILED DESCRIPTION OF THE PREFERRED EMBODIMENT

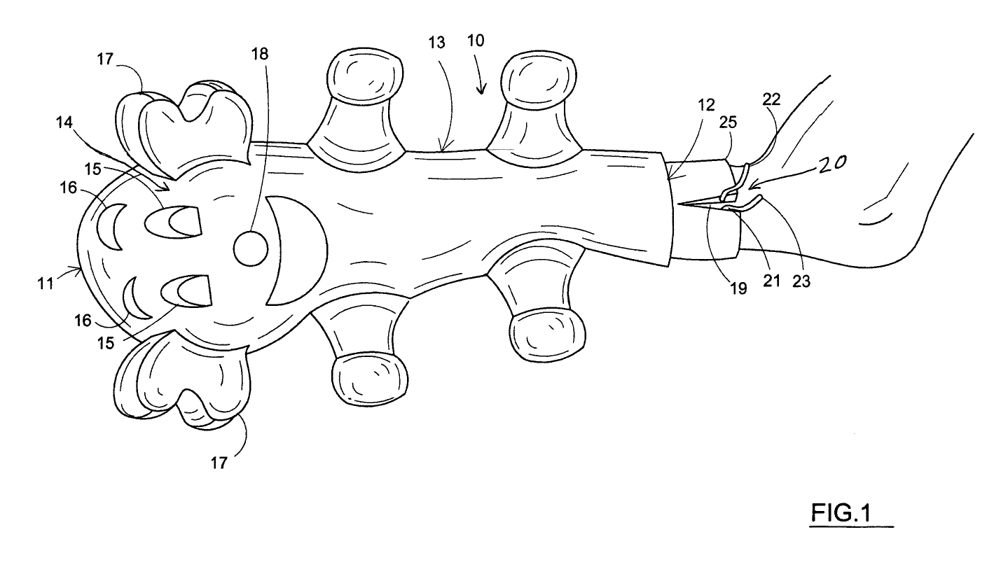

Referring to FIG. 1, the present invention is an elongated tubular sleeve 10 having a first proximal end 11 a second distal end 12 and a medial portion 13. Tube 10 is preferably flexible and of a soft material such as cotton or nylon, a blend thereof or any other material or combinations thereof that may be flame retardant.

First end 11 is preferably in the shape of an icon which may be an animal face 14 having a pair of eyes 15, eyebrows 16, ears 17 and a nose 18. The icon can be drawn or printed on tube 10 in two dimensions or as shown in the drawings the icon can be a 3-dimensional object attached to the tube 10 or the icon can be an integral part of the tube 10 or combinations thereof. There also may be more than one icon on tube 10. The object is for the icon such as face 14 which can be any face or any animal depiction to be friendly to encourage the user, child, to wear tube 10 at night when the child is ready to retire and sleep for the night to prevent the child from sucking the child's thumb. Typically an adult would place tube 10 on a child when it is desirable to prevent the child from sucking the child's thumb.

Second end 12 is an open end having an opening 20, of tube 10. Opening 20 may have a slit 19 to allow the opening 20 at end 12 to open a bit larger so that tube 10 can open sufficiently wide enough to allow a child/person to insert their hand into the inside of tube 10. Second end 12 has an overlapped collar portion 25 that forms a circular opening at the end of tube 10 that is threaded with a string 21 having two loose ends 22 and 23. String 21 can be pulled so that slit 19 is closed and end 12 conforms closely to the child's/person's forearm and then loose ends 22 and 23 are tied so that tube 10 is held securely in place on the child's arm, hopefully for the rest of the night while the child sleeps. An adult or other person putting the tube 10 on the user would then tied the loose ends 22 and 23 so that the tube 10 cannot be removed. Alternatively an elastic string 24 can be used to close the open end 12 around the user's forearm.

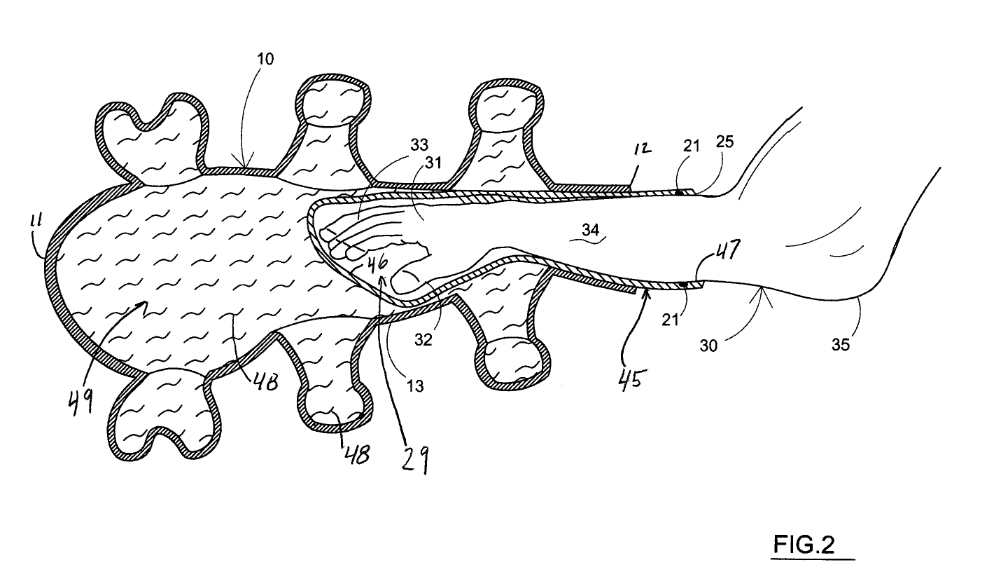

Referring to FIG. 2 the placement of the child's arm 30 can be seen within tube 10, where hand 31, fingers 33 and forearm 34 are seen within an inner tubular sleeve liner 45 having a first end 46 and a second open end 47. Overlapped portion 25 is a part of and the distal end of inner tube 45. In the preferred position the child's hand 31 will be in a compartment 29, at or close to or in contact with first end 46 and close to first end 11 with the thumb 32 and fingers 33 in contact with first end 46 and at or close to first end 11 of tube 10. Compartment 29 is preferably a single compartment allowing the user's hand to be free without restrictions of finger and/or thumb coverings such as a glove, so that the fingers or thumb are not enclosed within appendages typical with a glove type device. When the user's hand is in the compartment, it is more difficult for the user to locate the user's thumb to attempt to thumb suck. The child's forearm 34 is within tube 10 at the medial portion 13 and second end 12 is preferably at the forearm 34 of the child, close to the elbow 35. The inner tube 45 may extend further into tube 10 towards first end 11 than shown in FIG. 2. A padding 48 may fill the inner area between inner tube 45 and tube 10.

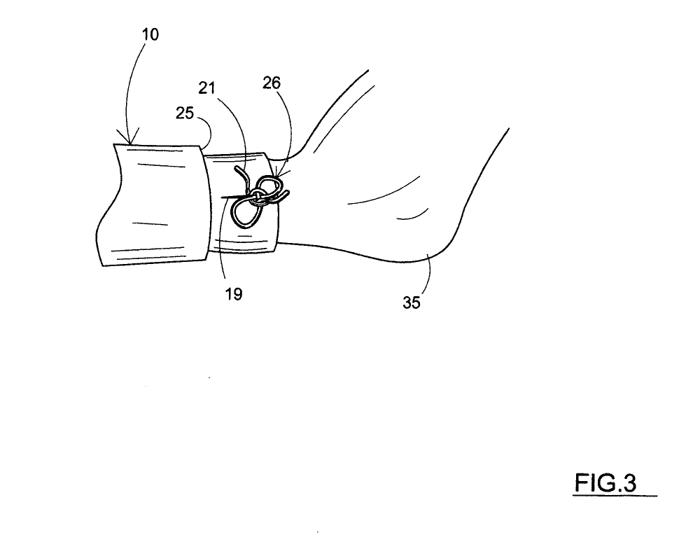

As seen if FIG. 3 slit 19 is drawn closed and string 21 is tied in a bow 26 closing end 12 around the forearm 34 of the user.

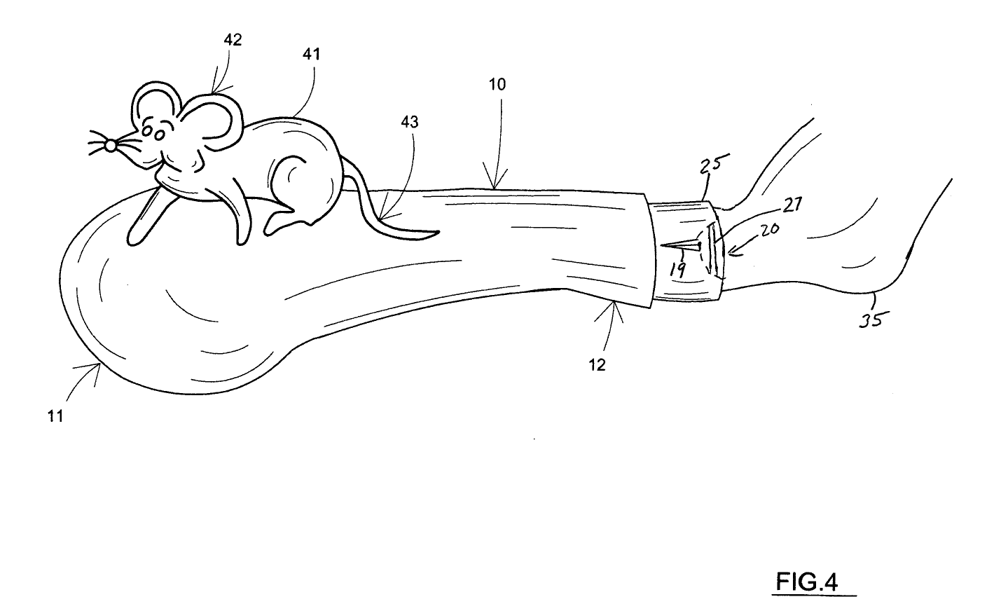

The design and shape of tube 10 can vary, as seen in FIG. 4, an animal icon 40, such as a mouse 41 having a head 42 and a tail 43 may be atop the closed end 11. The material of tube 10 may be in the form of animal fur and may not be smooth. FIG. 4 shows an alternate closure of overlapped portion 25, having an elastic band 27.

Tube 10 may or may not have an icon 40 and alternatively a design can be incorporated at the first end 11 of tube 10. Tube 10 may or may not have appendages 28. The shape of tube 10 may be tubular as shown or alternate shapes in keeping with the spirit of the invention.

The present invention is an apparatus to prevent thumb sucking for attachment to a user's arm having an elongated tubular sleeve of a soft pliable material having a first closed end and a second open end, a soft pliable material liner within said elongated tubular sleeve having a first liner closed end and a second liner open end, said liner constructed and arranged to receive the hand and forearm of a user, said second liner open end forming a collar for closure around a portion of a user's arm, said collar having a string for securing said collar to a user's arm and the exterior portion of said elongated tubular sleeve having an icon thereon, and that icon is either a two dimensional drawing on said tubular sleeve or a three dimensional icon or figure attached to said tubular sleeve.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. An apparatus to prevent thumb sucking for attachment to a user's arm comprising:
an elongated tubular sleeve of a soft pliable material having a first closed end and a second open end; a soft pliable material liner within said elongated tubular sleeve having a first liner closed end and a second liner open end; said liner constructed and arranged to receive the hand and forearm of a user; said second liner open end forming a collar for closure around a portion of a user's arm; said collar having a string for securing said collar to a user's arm;
the exterior portion of said elongated tubular sleeve having an icon thereon.

2. An apparatus as in claim 1, wherein said string is elastic.

3. An apparatus as in claim 1, wherein said icon is an animal shape attached to said tubular sleeve.

4. An apparatus as in claim 3, wherein said tubular sleeve has a plurality of icons.

5. An apparatus as in claim 1, wherein said icon is drawn or printed on said tubular sleeve.
